(19) United States
(12) Patent Application Publication
(10) Pub. No.: US 2019/0038209 A1
(43) Pub. Date: Feb. 7, 2019

(54) Using Spectral CT to Diagnose Thyroid Nodules

(71) Applicant: BOARD OF REGENTS, THE UNIVERSITY OF TEXAS SYSTEM, Austin, TX (US)

(72) Inventor: Todd C. Soesbe, Dallas, TX (US)

(21) Appl. No.: 16/071,031

(22) PCT Filed: Jan. 31, 2017
(86) PCT No.: PCT/US17/15783

§ 371 Date: Jul. 18, 2018

Related U.S. Application Data
(60) Provisional application No. 62/289,607, filed on Feb. 1, 2016.

(51) Int. Cl. A61B 5/00; A61K 33/24; A61K 51/04; A61B 6/03; G01J 3/28

(52) U.S. Cl. CPC A61B 5/4227; A61K 33/24; G01J 3/2823; A61B 6/03; A61K 51/04

(57) ABSTRACT

The present invention includes a method of measuring thyroid function comprising identifying a subject in need of measuring thyroid function or structure; providing the subject with an amount of an organified iodine sufficient to cause thyroid follicular cell uptake; and obtaining sufficient spectral computed tomography images of the iodine uptake in thyroid cells over an amount of time sufficient to determine thyroid cellular function.

### TECHNICAL FIELD OF THE INVENTION

The present invention relates in general to the field of thyroid function, and more particularly, to novel methods for using Spectral CT to diagnose thyroid nodules.

### BACKGROUND OF THE INVENTION

Without limiting the scope of the invention, its background is described in connection with computed tomography (CT).

U.S. Pat. No. 8,761,479, entitled, “System and method for analyzing and visualizing spectral CT data”, is directed to a system and method for analyzing and visualizing spectral CT data that includes access to a set of image data acquired from a patient comprising spectral CT data, identifying one or more target regions of interest (TROIs) and a reference region of interest (RROI) from the set of image data, extracting of a plurality of target spectral Hounsfield unit (HU) curves from image data representing the plurality of TROIs, extracting a reference spectral HU curve from image data representing the RROI, normalizing the plurality of target spectral HU curves with respect to the reference spectral HU curve, and displaying of the plurality of normalized target spectral HU curves.

U.S. Pat. No. 8,391,439, entitled, “Detector array for spectral CT” is directed to a radiation detector that includes a two-dimensional array of upper scintillators, which is disposed facing an x-ray source to convert lower energy radiation events into visible light and transmit higher energy radiation. The patent provides that a two-dimensional array of lower scintillators is disposed adjacent the upper scintillators distally from the x-ray source to convert the transmitted higher energy radiation into visible light, and that upper and lower photodetectors are optically coupled to the respective upper and lower scintillators at an inner side of the scintillators.

U.S. Patent Application Publication No. 2015/0117593, entitled Method for spectral CT local tomography” is directed to a method for performing reconstruction for a region of interest (ROI) of an object by designating the ROI within the object, the ROI being located within a scan field of view (FOV) of a combined third- and fourth-generation CT scanner, the CT scanner including fixed photon-counting detectors (PCDs), and an X-ray source that rotates about the object in synchronization with a rotating detector. The method is also said to teach determining each PCD based on a size and location of the designated ROI, and further performing a scan to obtain a first data set from the rotating detector and a second data set from the plurality of PCDs, while turning each PCD on and off according to the determined schedule, and displaying the results of the same.

### SUMMARY OF THE INVENTION

In one embodiment, the present invention includes a method of measuring thyroid function comprising: identifying a subject in need of measuring thyroid function or structure; providing the subject with an amount of an organified iodine sufficient to cause thyroid follicular cell uptake; and obtaining sufficient spectral computed tomography images of the iodine uptake in thyroid cells over an amount of time sufficient to determine thyroid cellular function. In one aspect, the thyroid is suspect of having at least one of a thyroid nodule, euthyroidism, hyperthyroidism, hypothyroidism, thyroid cancer metastasis, thyroid cancer recurrence, or thyroid disease. In another aspect, the organified iodine is provided to the subject orally, intravenously, intraperitoneally, enterally, parenterally, or subcutaneously. In another aspect, the organified iodine is potassium iodide. In another aspect, the thyroid tissue or cellular function is determined without the need for radioactive isotopes. In another aspect, the Spectral CT scan is capable of providing at least one of higher spatial resolution, a cross-sectional image, or a 3D rendering of thyroid tissue or thyroid cells. In another aspect, the method further comprises the step of determining a pre-scan thyroid iodine base line of 1 to 2 mg/mL in thyroid tissue before providing the organified iodine to the subject. In another aspect, the method further comprises the step of displaying an iodine map of the thyroid or thyroid tissue of the subject. In another aspect, the method further comprises the step of displaying one or more contrast enhanced structures of the thyroid or thyroid tissue without calcium. In another aspect, the method further comprises the step of providing the subject with thyrotropin alfa prior to, concurrently with, or after providing the subject with the organified iodine to increase uptake of iodine in the thyroid. In another aspect, the method further comprises the step of obtaining a spectral CT scan and measuring at least one of incidental thyroid nodules (ITN), benign thyroid nodules, thyroid cancer nodules, a thyroid cancer malignancy, or a thyroid cancer recurrence. In another aspect, the method further comprises the step of obtaining a thyroid function scan at a first time before treatment of a thyroid cancer, and obtaining a second thyroid function scan after a pre-determined time following treatment, and comparing the function at the two or more times to determine the effect of the treatment of thyroid function. In another aspect, the image of the thyroid is not phase dependent.

Another embodiment of the present invention includes a method of measuring thyroid function comprising identifying a subject in need of measuring thyroid function; providing the subject with an amount of an organified iodine sufficient to measure the presence of iodine in thyroid follicular cells in the thyroid tissue; and obtaining sufficient spectral computed tomography images of the iodine in the thyroid follicular cells for an amount of time sufficient to measure thyroid tissue or thyroid cellular function. In one aspect, the thyroid is suspect of having at least one of a thyroid nodule, euthyroidism, hyperthyroidism, hypothroidism, thyroid cancer metastasis, thyroid cancer recurrence, or thyroid disease. In another aspect, the organified iodine is provided to the subject orally, intravenously, intraperitoneally, enterally, parenterally, or subcutaneously. In another aspect, the organified iodine is potassium iodide. In another aspect, the thyroid tissue or cellular function is determined without the need for radioactive isotopes. In another aspect, the Spectral CT scan is capable of providing at least one of higher spatial resolution, a cross-sectional image, or a 3D rendering of thyroid tissue or thyroid cells. In another aspect, the method further comprises the step of determining a pre-scan thyroid iodine base line of 1 to 2 mg/mL in thyroid tissue before providing the organified iodine to the subject. In another aspect, the method further comprises the step of displaying an iodine map of the thyroid or thyroid tissue of the subject. In another aspect, the method further comprises the step of displaying one or more contrast enhanced structures of the thyroid or thyroid tissue without calcium. In another aspect, the method further comprises the step of providing the subject with thyrotropin alfa prior to, concurrently with, or after providing the subject with the organified iodine to increase uptake of iodine in the thyroid. In another aspect, the method further comprises the step of obtaining a spectral CT scan and measuring at least one of incidental thyroid nodules (ITN), benign thyroid nodules, thyroid cancer nodules, a thyroid cancer malignancy, or a thyroid cancer recurrence. In another aspect, the method further comprises the step of obtaining a thyroid function scan at a first time before treatment of a thyroid cancer, and obtaining a second thyroid function scan after a pre-determined time following treatment, and comparing the function at the two or more times to determine the effect of the treatment of thyroid function. In another aspect, the image of the thyroid is not phase dependent. In another aspect, the image of the thyroid is at the cellular or subcellular level.

Yet another embodiment is a method of evaluating a candidate drug believed to be useful in treating a disease state of thyroid tissue, the method comprising: a) measuring the amount of iodine uptake in thyroid follicular cells from tissue suspected of having one or more thyroid nodules, benign thyroid nodules, thyroid cancer nodules, thyroid cancer malignancies, or thyroid cancer recurrence from a set of patients; b) administering a candidate drug to a first subset of the patients, and a placebo to a second subset of the patients; c) repeating step a) after the administration of the candidate drug or the placebo; and d) determining if the candidate drug reduces the number of thyroid nodules, benign thyroid nodules, thyroid cancer nodules, thyroid cancer malignancies, or thyroid cancer that is statistically significant as compared to any reduction occurring in the second subset of patients, wherein a statistically significant reduction indicates that the candidate drug is useful in treating said disease state.

### BRIEF DESCRIPTION OF THE DRAWINGS

For a more complete understanding of the features and advantages of the present invention, reference is now made to the detailed description of the invention along with the accompanying figures and in which:

FIG. 1 plots the theoretical thyroid iodine uptake versus oral KI dose (30% uptake in 24 hours) for four different total thyroid volumes.

FIG. 2 shows a conventional CT image of the prior art that shows a CT cross-sectional image of euthyroid (top) and nodule (bottom) patients 24 hours after oral KI.

FIG. 3 is a comparison figure that shows a coronal iodine map of the renal cortex acquired with our Philips iQon Spectral CT system using the present invention (arterial phase, iopamidol IV injection).

FIG. 4 is a graph that shows an iodine map of a euthyroid patient with IV contrast only.

FIG. 5 shows before and after images as follows: Left panels: shows iodine-no-water images (i.e., iodine map) of the rat thyroid before oral potassium iodide administration. The two lobes of the thyroid can clearly be seen with iodine levels measuring approximately 1.5 mg I/mL. Right panels: shows iodine maps of the same rat 10 hours after 654 mg of oral potassium iodide. The iodine levels within the thyroid have increased by approximately 100% (i.e., doubled). Notice the increased intensity of the right thyroid lobe when comparing before and after images (white arrows). Note that the window and leveling are the same for each image.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Spectral CT (also known as dual-energy, dualsource, and dual-detector CT) can accurately measure the concentration of iodine within tissue. This concentration is given in units of mg of Iodine per mL (mg/mL). Methods for using Spectral CT to help diagnose renal lesions as malignant or benign are currently being explored. These methods rely upon the intravenous injection of approximately 100 mL of an iodinated contrast agent (e.g., Iopamidol) during the CT scan with the purpose being to quantify the degree of vasculature within the renal lesions. The present invention uses Spectral CT to diagnose thyroid lesions (nodules) and other thyroid diseases. In the present method, rather than using intravenous iodine contrast to evaluate the extracellular vasculature of the lesion, the inventors use thyroid follicular cell uptake of orally administered potassium iodide (K1) to create targeted CT contrast. By using the intracellular uptake of non-radioactive iodine the method taught herein can use Spectral CT to image and measure the function of thyroid tissue in a manner similar to Iodine-123 Scintigraphy and Iodine-131 SPECT/CT, with a marked improvements in spatial resolution and lesion localization. Thus, this method combines for the first time the convenience and high-resolution of CT with the functional imaging of Nuclear Medicine techniques.

The method of the present invention offers significant advantages over Iodine-123 Scintigraphy in two main ways: (1) the method provides a much higher spatial resolution (sub-millimeter versus 3 to 5 millimeter), and (2) the method offers true 3-D functional imaging as opposed to the 2-D images offered by scintigraphy. Both factors would help with thyroid nodule localization and tissue specificity. Further, the scan time for CT is also much faster than Scintigraphy, which improves not only the patient experience, but also patient throughput. Also, the procedure for administering non-radioactive potassium iodide would be much simpler and less expensive than that for radioactive Iodine-125.

Unlike other recent methods of tissue iodine quantification using Spectral CT to measure thyroid disease rely upon either: (A) the natural endogenous levels of intracellular iodine (i.e., 0.5 to 2.0 mg/mL), or (B) the uptake of exogenous Iodinated contrast agents (e.g., Iopamidol) to differentiate between malignant and benign lesions. The method of the present invention differs from prior methods by using the thyroid follicular cell uptake of orally administered potassium iodide (over a 24 hour period) to increase the signal-to-noise ratio of the thyroid tissue far above the surrounding background tissue. In this manner the functional information is amplified and not lost by intravascular iodine administration. Further, the improved signal-to-noise ratio helps overcome beam-hardening artifacts (i.e., image noise) caused by the clavicle bone and cervical spine vertebrae, allowing for a more accurate Iodine quantification. Also, the method of the present invention provides real-time of thyroid functional imaging would offer a much higher spatial resolution than current nuclear medicine techniques (i.e., Iodine-123 Scintigraphy and Iodine-131 SPECT/CT).

Finally, using non-radioactive potassium iodide as a contrast agent would reduce the risk of secondary cancers (e.g., colon and leukemia) caused by beta particle emissions from Iodine-131 SPECT/CT. Also, the improved spatial-resolution of Spectral CT allows for better localization of primary and recurring thyroid cancers than Iodine-131 SPECT/CT.

Using Spectral CT and organified iodine for enhanced diagnosis of thyroid nodules.

Spectral CT represents the next generation of CT technology and improves upon conventional CT by also providing differentiation and quantification of certain endogenous and exogenous materials found within tissue including water, calcium, iron, uric acid, and iodine (1). Spectral CT allows for enhanced diagnosis by going beyond the conventional grayscale images of total linear attenuation to images representing the densities of specific materials, which can be displayed in units of mg/mL or Hounsfield units. With some limitations, Spectral CT is capable of identifying what materials are present in a voxel and how much of each material is present. The data provided by Spectral CT offers new metrics for disease detection and diagnosis, particularly for indeterminate cases, without any increase in radiation exposure to the patient.

The present invention uses Spectral CT to help diagnose thyroid nodules as malignant or benign. The present inventors determined, using the method of the present invention, that the mg/mL levels of organified iodine, as measured and imaged by Spectral CT, can be used as a biomarker for improved diagnosis. This method is supported by a recent ex vivo study showing a correlation between iodine density and thyroid nodule pathology (2). The Spectral CT contrast in the thyroid can be enhanced by orally administering non-radioactive potassium iodide (KI) 24 hours before the scan, similar to RAIU. High-resolution cross-sectional and 3D images of the organified iodine within the thyroid can then be created using, e.g., a Philips iQon Spectral CT system. The Philips iQon Spectral CT system is able to look at the iodine signal, exclusively, excluding all other tissues like bone, fat, muscle, and water. This system can also accurately quantify the levels of iodine present in the thyroid in units of mg/mL, a feature unique to Spectral CT.

Current applications of Spectral CT to thyroid disease use intravascularly (IV) administered CT contrast agents like iopamidol (3).

The method of the present invention differs by using the organification of orally administered iodine in the form of KI as a thyroid targeted contrast agent. The IV contrast method, although similar to that used for renal lesions, is flawed for thyroid applications because it is non-targeted, does not offer any functional information, and is highly CT-phase dependent. Furthermore, imaging the organified iodine with Spectral CT produces true functional images of the thyroid, instead of only imaging the vascularity and extracellular space as with IV contrast. In this manner, images of the thyroid function similar to those obtained from ¹²³I- or ¹³¹I RAIU scintigraphy are acquired, but at much a higher spatial resolution (0.625 mm isotropic) and with true cross-sectional and 3D rendering capabilities. This is particularly advantageous when imaging multi-nodular patients where each nodule can be easily identified and the iodine densities measured separately.

Methods. The present invention can be used to measure nodular, multi-nodular, hyperthyroid, hypothyroid, and euthyroid patients (3 to 5 each). A pre-contrast Spectral CT scan can determine the baseline iodine densities, which are typically 1.0±0.5 mg/mL for euthyroid tissue (2). Patients can then be given five 130 mg KI pills (500 mg iodine total) to take orally. After 24 hours of uptake, the patients can be scanned a second time and the changes in iodine densities at several locations within the thyroid and nodules will be measured. These iodine densities can then be correlated with pathology for patients undergoing resection. Although not likely required, in some cases it may be beneficial to use thyrogen to increase the iodine uptake in the thyroid.

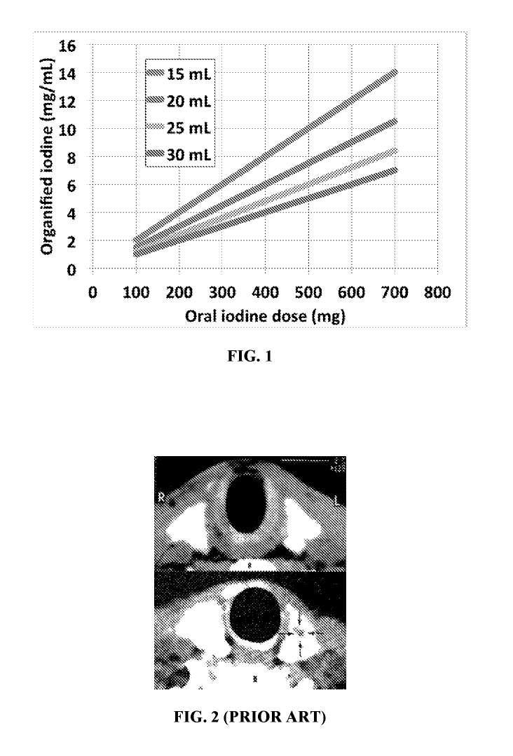

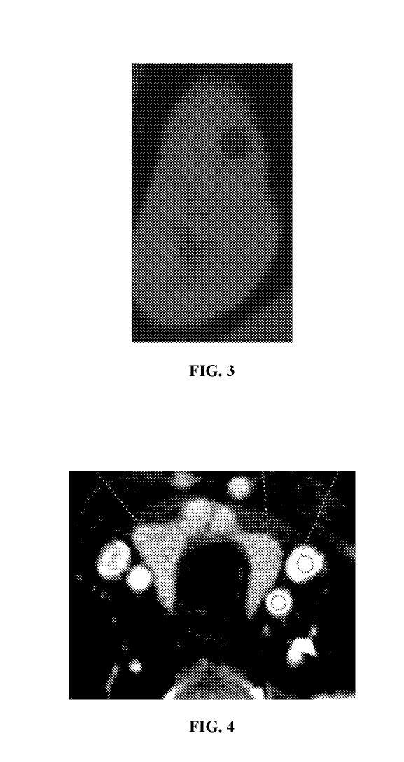

FIG. 1 plots the theoretical thyroid iodine uptake versus oral KI dose (30% uptake in 24 hours) for four different total thyroid volumes (legend). A 500 mg dose of iodine (five 130 mg KI pills) yields an organified iodine density from 5 to 10 mg/mL. This iodine density is well above the 0 to 0.5 mg/mL background of non-thyroid tissue and is readily imaged and accurately quantified by Spectral CT (FIG. 3). This dose is similar to other daily oral doses used clinically (375 mg iodine from Lugol's 5%, and 750 mg iodine from SSKI).

To provide a comparison, FIG. 2 shows conventional CT cross-sectional images of euthyroid (top) and nodule (bottom) patients from the prior art (4), displaying enhanced thyroid contrast 24 hours after a 500 mg oral dose of iodine. Note that since the contrast is from organified iodine there is no contrast in the carotid artery or jugular vein as seen with vascular CT contrast (3). Also, in the prior art, the small 3 mm hypo-intense nodule is easily detected in the left lobe of the bottom image (arrows). These images show that low doses of oral KI can be used to enhance thyroid contrast and measure thyroid function.

The method and system used with the present invention, not only allows much higher quality functional images, it also provides functional iodine density measurements (mg/mL) as a biomarker. FIG. 3 shows a coronal iodine map of the renal cortex acquired with our Philips iQon Spectral CT system (arterial phase, iopamidol IV injection). The iodine density measured in the cortex is 5.6 mg/mL and gives excellent contrast to the 10 mm diameter hypo-intense renal lesion (0.2 mg/mL) simulating a “cold” thyroid nodule.

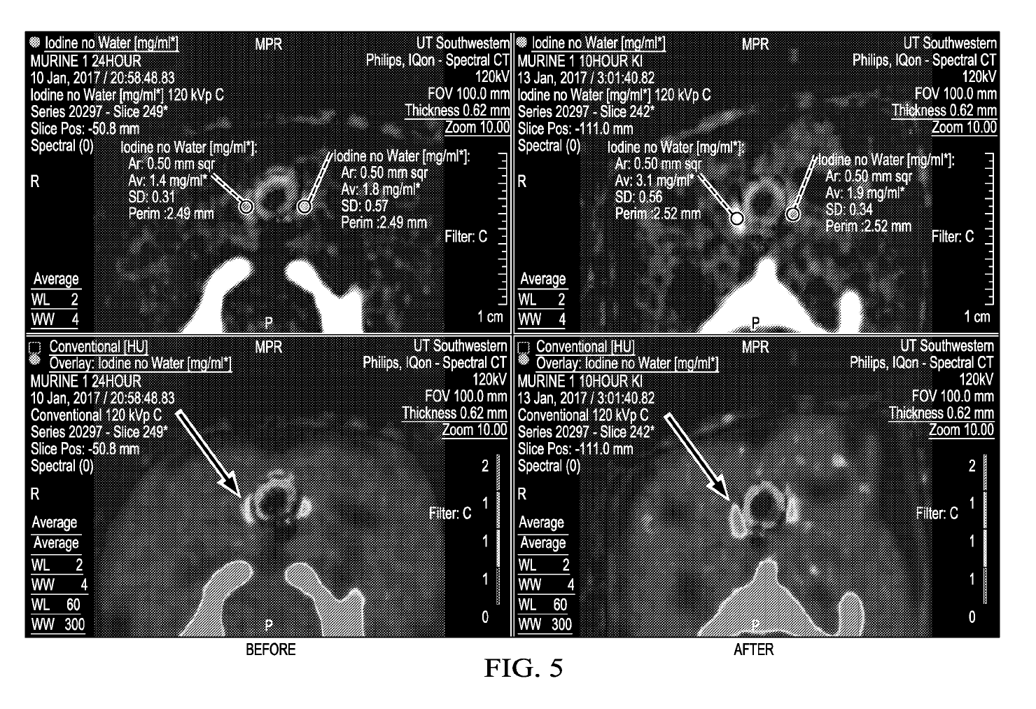

FIG. 4 shows a cross-sectional iodine map of a euthyroid patient with IV contrast taken with our Philips iQon Spectral CT. The thyroid iodine density measured 4.9 mg/mL and simulates what the contrast would be after 500 mg of oral iodine.

Next, a Philips IQon Spectral CT scanner was used to obtain in vivo animal data. Three healthy female Fischer rats ranging from 150 g to 250 g mass were each anesthetized using 2.5% isoflurane and scanned in a supine position using the highest resolution settings for the IQon Spectral CT clinical scanner (0.625 mm slice thickness with 0.20 mm in-plane pixel resolution). The baseline endogenous iodine levels within the rat thyroid were measured using the iodine-no-water map and averaged 1.5 mg I/mL. These levels are similar to the baseline endogenous iodine levels found within healthy human thyroid tissue. The rats were then each given a bolus dose of 654 mg of potassium iodide (equivalent to 500 mg of iodine) that was dissolved in 1 mL of water and administered orally using a gavage. After 10 hours of uptake the rats were again anesthetized with 2.5% isoflurane and scanned a second time on the IQon Spectral CT using the same settings as before. The iodine levels within the rat thyroid now averaged 3.0 mg I/mL, or a 100% increase in iodine. The data in FIG. 5 show that safe low doses of oral potassium iodide can be used to significantly increase the intracellular (i.e., organified) iodine levels found within thyroid tissue. Generally, a 500 mg dose of oral iodine given to humans can increase the level of organified iodine in the thyroid tissue (after a full 24 hours of uptake) from 1.5 mg I/mL to 5-10 mg I/ml depending on total thyroid volume. These increased iodine levels would not only improve thyroid contrast when imaged with Spectral CT, but more importantly, they can also be used as a biomarker to determine if thyroid nodules are malignant or benign.

FIG. 5 shows before and after images as follows: Left panels: shows iodine-no-water images (i.e., iodine map) of the rat thyroid before oral potassium iodide administration. The two lobes of the thyroid can clearly be seen with iodine levels measuring approximately 1.5 mg I/mL. Right panels: shows iodine maps of the same rat 10 hours after 654 mg of oral potassium iodide. The iodine levels within the thyroid have increased by approximately 100% (i.e., doubled). Notice the increased intensity of the right thyroid lobe when comparing before and after images (white arrows). Note that the window and leveling are the same for each image.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

(1) Marin D, et al. Radiology 2014; 271:327-342.
(2) Li M, et al. Invest Radiol 2012; 47:58-64.
(3) Lau D, et al. Am J Neuroradiol 2013; 34:E91-E93.
(4) Vette J K, Acta Endocrin 1985; 268:1-82.

1. A method of measuring thyroid function comprising:
identifying a subject in need of measuring thyroid function or structure;
providing the subject with an amount of an organified iodine sufficient to cause thyroid follicular cell uptake; and
obtaining sufficient spectral computed tomography images of the iodine uptake in thyroid cells over an amount of time sufficient to determine thyroid cellular function.

2. The method of claim 1, wherein the thyroid is suspect of having at least one of a thyroid nodule, euthyroidism, hyperthyroidism, hypothroidism, thyroid cancer metastasis, thyroid cancer recurrence, or thyroid disease.

3. The method of claim 1, wherein the organified iodine is provided to the subject orally, intravenously, intraperitoneally, enterally, parenterally, or subcutaneously.

4. The method of claim 1, wherein the organified iodine is potassium iodide.

5. The method of claim 1, wherein the thyroid tissue or cellular function is determined without the need for radioactive isotopes.

6. The method of claim 1, wherein the Spectral CT scan is capable of providing at least one of higher spatial resolution, a cross-sectional image, or a 3D rendering of thyroid tissue or thyroid cells.

7. The method of claim 1, further comprising the step of determining a pre-scan thyroid iodine base line of 1 to 2 mg/mL in thyroid tissue before providing the organified iodine to the subject.

8. The method of claim 1, further comprising the step of displaying an iodine map of the thyroid or thyroid tissue of the subject.

9. The method of claim 1, further comprising the step of displaying one or more contrast enhanced structures of the thyroid or thyroid tissue without calcium.

10. The method of claim 1, further comprising the step of providing the subject with thyrotropin alfa prior to, concurrently with, or after providing the subject with the organified iodine to increase uptake of iodine in the thyroid.

11. The method of claim 1, further comprising the step of obtaining a spectral CT scan and measuring at least one of incidental thyroid nodules (ITN), benign thyroid nodules, thyroid cancer nodules, a thyroid cancer malignancy, or a thyroid cancer recurrence.

12. The method of claim 1, further comprising the step of obtaining a thyroid function scan at a first time before treatment of a thyroid cancer, and obtaining a second thyroid function scan after a pre-determined time following treatment, and comparing the function at the two or more times to determine the effect of the treatment of thyroid function.

13. The method of claim 1, wherein the image of the thyroid is not phase dependent.

14. A method of measuring thyroid function comprising:
identifying a subject in need of measuring thyroid function;
providing the subject with an amount of an organified iodine sufficient to measure the presence of iodine in thyroid follicular cells in the thyroid tissue; and
obtaining sufficient spectral computed tomography images of the iodine in the thyroid follicular cells for an amount of time sufficient to measure thyroid tissue or thyroid cellular function.

15. The method of claim 14, wherein the thyroid is suspect of having at least one of a thyroid nodule, euthyroidism, hyperthyroidism, hypothroidism, thyroid cancer metastasis, thyroid cancer recurrence, or thyroid disease.

16. The method of claim 14, wherein the organified iodine is provided to the subject orally, intravenously, intraperitoneally, enterally, parenterally, or subcutaneously.

17. The method of claim 14, wherein the organified iodine is potassium iodide.

18. The method of claim 14, wherein the thyroid tissue or thyroid cellular function is determined without the use of radioactive isotopes.

19. The method of claim 14, wherein the Spectral CT scan is capable of providing at least one of higher spatial resolution, cross-sectional images, or 3D renderings of thyroid tissue or thyroid cells.

20. The method of claim 14, further comprising the step of determining a pre-scan thyroid iodine base line of 1 to 2 mg/mL in thyroid tissue before providing the organified iodine to the subject.

21. The method of claim 14, further comprising the step of displaying an iodine map of the thyroid of the subject.

22. The method of claim 14, further comprising the step of displaying one or more contrast enhanced structures of the thyroid without calcium.

23. The method of claim 14, further comprising the step of providing the subject with thyrotropin alfa prior to, concurrently with, or after providing the subject with the organified iodine to increase uptake of iodine in the thyroid.

24. The method of claim 14, further comprising the step of obtaining a spectral CT scan and measuring at least one of incidental thyroid nodule (ITN) diagnosis, benign thyroid nodules, thyroid cancer nodules, thyroid cancer malignancies, or thyroid cancer recurrence.

25. The method of claim 14, further comprising the step of obtaining a thyroid function scan at a first time before treatment of a thyroid cancer, and obtaining a second thyroid function scan after a pre-determined time following treatment, and comparing the function at the two or more times to determine the effect of the treatment of thyroid function.

26. The method of claim 14, wherein the image of the thyroid is not phase dependent.

27. The method of claim 14, wherein the image of the thyroid is at the cellular or subcellular level.

28. (canceled)
